(19) United States
(12) Patent Application Publication
(10) Pub. No.: US 2005/0238611 A1
(43) Pub. Date: Oct. 27, 2005

(54) Cosmetic compositions and their use

(75) Inventors: Pietro Rando, Opera (MI) (IT); Giuseppe Maio, Zelo Surrigone (MI) (IT)

Correspondence Address: AKERMAN SENTERFITT, P.O. BOX 3188, WEST PALM BEACH, FL, 33402-3188, US

(73) Assignee: Intercos S.p.A., Milano (IT)

(21) Appl. No.: 11/024,612

(22) Filed: Dec. 29, 2004

(30) Foreign Application Priority Data
Apr. 23, 2004 (EP) 04425281.5

(51) Int. Cl. A61K 7/06; A61K 7/11

(52) U.S. Cl. 424/70.122

(57) ABSTRACT

The present invention relates to a cosmetic composition particularly for the make-up of the facial skin, the lips and the eyelashes. It contains as essential ingredients a particular silicone-containing polyurethane. The particular silicone, used as pre-polymer in the polyurethane of the invention is an alkoxylated, bis-hydroxyalkyl group terminated polydialkylsiloxane, in which the reactive OH— group is attached to a carbon atom. The balance contains the usual cosmetic excipients, colorants and other cosmetic additives, particularly clays, waxes and solvents. By the use of the particular silicone-containing polyurethane, improved film-forming properties of the composition are obtained, and the composition is transfer-resistant and easy to apply and skin friendly and comfortable in use. By introducing different kind of chain extenders into the silicone-containing polyurethanes, their physical, rheological and cosmetic properties can be tuned according to the formulator's needs.

### FIELD OF THE INVENTION

The present invention relates to cosmetic compositions comprising a silicone-containing polyurethane and to a process for preparing said silicone-containing polyurethane.

More particularly it relates to a cosmetic composition comprising a particular class of silicone-containing polyurethanes with particular cosmetic properties, especially for the skin, hair, nails, lips, eyelashes etc, and to a process for preparing said silicone-containing polyurethanes.

### BACKGROUND OF THE INVENTION

Cosmetic products for the make-up of the face, the lips, the eyelashes etc often suffer from the drawback that, when they come into contact with e.g. the fingers or clothing, they tend to smudge or soil these surfaces. In addition, in some cases the make-up can also appear to be not homogeneous. Consequently, in order to avoid these problems cosmetic products which have high adhesive properties and which provide for the deposition of a homogeneous, long-lasting film onto e.g. the facial skin, the lips, the eyelashes etc are of particular interest.

Usually, the formulator attempts to achieve these properties by including a functional film-forming polymer in the cosmetic product. Such polymer must also be physiologically compatible with the skin and offer protection against dehydration, UV light and so on.

However, the Applicant has found that commonly used functional film-forming polymers proved to be unsuitable for application to the lips, as the film tended to crack down under the normal and continuous lips' movement. Moreover, toughness of the film often caused a feeling of discomfort when applied onto the lips or onto the skin.

It is, therefore, an object of the present invention to overcome these drawbacks and to provide a cosmetic composition comprising a particular silicone-containing polyurethane with improved film-forming, adhesive and sensorial properties.

### DESCRIPTION OF THE RELATED ART

It has already been proposed in the art to use silicone-containing polyurethanes as film-forming polymers in cosmetic compositions for the skin, hair, eyelashes etc. Thus, U.S. Pat. No. 6,166,093 (Mougin, et al.) describes the use of a polyurethane block polycondensation product comprising a polysiloxane graft for treating keratinous materials. The polysiloxane oligomer has a diol or diamine function at only one end of its chain.

A similar technology is described in U.S. Pat. No. 6,319,959(Mougin, et al.).

In U.S. Pat. No. 5,643,581 (Mougin, et al.) a cosmetic composition for the skin, hair, lips etc is described which contains a multiblock polycondensate of a polyurethane and a polysiloxane, wherein the polyurethane further comprises anionic or cationic groups.

In U.S. Pat. No. 2,002,076425 (=FR 2,814,365; Mondet, et al.) a cosmetic composition is described which contains a polyurethane with at least two urethane groups and at least one hydrocarbon-based unit chosen from hydrocarbon blocks and grafts or esters thereof. These polymers may additionally contain a polyorganosiloxane.

Finally, dimethiconol- or dimethicone-containing polyurethanes are known cosmetic ingredients, see e.g.U.S. Pat. No. 2,002,0028875 (Anderle, et al.) and U.S. Pat. No. 6,120,753 (Vinski, et al.). Dimethiconol-containing polyurethanes are commercially available from ALZO, Inc under the tradename Polyderm PPI-SI.

Despite these prior proposals there is still a need for a cosmetic composition having high film-forming and adhesive properties together with improved sensorial properties of smoothness and comfort, and it is an object of the present invention to provide such a composition.

### SUMMARY OF THE INVENTION

According to the present invention, this object is achieved by providing a cosmetic composition comprising a particular silicone-containing polyurethane with a molecular weight of between 5,000 and 800,000, preferably between 40,000 and 400,000, and particularly preferably between 100,000 and 280,000. The particular silicone, used as pre-polymer in the polyurethane of the invention is an alkoxylated, bis hydroxyalkyl group terminated polydialkylsiloxane, in which the reactive OH— group is attached to a carbon atom.

The alkyl group in the polydialkylsiloxane may be a methyl, ethyl, propyl or butyl group, of which the methyl group is preferred. The terminal hydroxyalkyl group may be a hydroxymethyl, hydroxyethyl, hydroxypropyl, hydroxybutyl group, of which the hydroxyethyl group is preferred and the alkoxyl moiety may be a methoxyl, ethoxyl, propoxyl or butoxyl moiety, of which the propoxyl moiety is preferred.

The silicone pre-polymer may contain from 1 to 8, preferably from 1 to 5 alkoxy groups, which may be methoxy-, ethoxy-, propoxy- and butoxy-groups and mixtures thereof. Ethoxy-groups are preferred.

A preferred silicone pre-polymer according to the present invention is α,ω-di(2-propoxyethanol) polydimethylsiloxane (=polydimethylsiloxane, 2-propoxyethanol terminated), having about 20 silyloxy units in its chain.

A preferred polyurethane according to the present invention, having a molecular weight of between 100,000 and 280,000, is the reaction product of this preferred silicone pre-polymer with isophorone diisocyanate

### DETAILED DESCRIPTION OF THE INVENTION

The amount of the silicone-containing polyurethane used in the cosmetic compositions ranges from 0.25 to 40% by weight of the composition, preferably from 0.5 to 30% and particularly preferably from 1 to 20%.

The polyurethane of the invention can be suitably prepared by methods, known in the art for the manufacture of polyurethanes. Such methods involve the reaction between a siliconol and a diisocyanate in a solvent in the presence of a catalyst.

Another method is the so-called two-steps route, in which the first step is to produce a precursor of the final polymer. This precursor has a more controllable reactivity and it is called a quasi-polymer. The final polymer is then produced by the reaction of the quasi-polymer with a chain extender, also known as a curative. Chain extenders are polyfunctional chemicals such as monoalcohols, poly-alcohols, dicarboxylic acids and so on. The chain extender zips up the quasi-polymer molecules, thus increasing the molecular weight and creating the final polymer. The chain extenders play an important role in the synthesis as many of the actual cosmetic and rheological properties of the polymer depend on the appropriate choice of the chain extender.

The polyurethanes of the present invention preferably contain a chain extender.

Pre-polymers

The pre-polymers, suitable for use in the manufacture of the polyurethanes of the present invention, are siliconols such as the alkoxylated polydimethylsiloxanes, already described above in the summary of the invention.

Diisocyanates

The diisocyanates, suitable for use in the manufacture of the polyurethanes of the present invention can be any diisocyanate which is known as raw material for polyurethanes, such as toluene diisocyanate, 2,2,4-trimethyl-1,6-hexamethylene diisocyanate, methylene-bis(4-cyclohexyl isocyanate), isophorone diisocyanate etc. The most suitable diisocyanates are isophorone diisocyanate, lysine alkyl ester diisocyanate and arginine alkyl ester diisocyanate, wherein the alkyl group may be methyl, ethyl, propyl and butyl and mixture thereof.

Chain Extenders

If the polyurethanes of the present invention also contain a chain extender—which is a preferred embodiment of the invention—the choice of the chain extender determines the characteristics of the polymer's chain and the behaviour in cosmetic formulations. Thus, simple diols yield linear polyurethanes, whilst triols or polyols create highly branched polyurethanes. The structure of the polyurethane has great influence on its physical behaviour when it is used in cosmetic formulations. Thus e.g. the viscosity of its solution can be tuned by varying the degree of branching.

The chain extender also carries in the polymer its whole structure besides the mere alcoholic or carboxylic functionality needed for the chain extension. This means that the molecule required as chain extender can be chosen according to the cosmetic properties which the formulator wishes to impart to a cosmetic product. Thus e.g. the lipophylicity of the polyurethane can be tuned by introducing different long carbon chain 1,2-diols or different fatty acid monoglycerides.

Particularly suitable chain extenders were found to be C8-C28 fatty acid esters of polyhydroxy compounds such as glycerol, polyglycerol, pentaerythritol, sucrose, glucose, fructose, sorbitan etc. A preferred extender of this class is sorbitan monostearate.

Other suitable extenders are esters of C8-C28 fatty alcohols with hydroxyacids such as lactic acid, maleic acid, tartaric acid, citric acid. Non-limitative examples are stearyl tartrate, C12-C13 alkyl malate, C12-C13 alkyl lactate, C14-C15 alkyl citrate.

Other suitable extenders were found to be acyl-aminoacids derived from glutamic acid, leucine, arginine, cysteine, lysine, serine, threonine, tyrosine, hydroxyproline, ornithine, citrulline, homocysteine, homoserine, cystine, statine with (C8-C28) acyl groups such as stearoyl glutamate and stearoyl leucine.

Yet another group of suitable extenders are vitamins as such or C2-C28 alkyl esters or ethers of vitamins, such as esters or ethers of vitamin B1, B2, B5, B6, and C.

Catalysts

In the above step-polymerization process a catalyst is desirable to shorten the reaction time. Well-known catalysts are tertiary amines such as DABCO (bicycloamine) or triethylamine, which work better on aromatic isocyanates. Metal Lewis acids, such as tin, bismuth, iron or zinc derivatives efficiently catalyze reaction with both aliphatic and aromatic isocyanates. We have found that for cosmetic purposes the best catalysts are zinc salts of long-chain(C8-C28) fatty acids (“zinc soaps”) such as zinc laurate, zinc myristate, zinc palmitate, zinc oleate, zinc linoleate, zinc linolenate and particularly zinc stearate. Zinc stearate has the advantage that for cosmetic purposes it does not have to be removed from the reaction mixture.

Reaction Solvent

The above polymerization process is usually carried out in a solvent. Many solvents are known for use in this process.

Light paraffins and isoparaffins with from 8-24 carbon atoms in their alkyl chain were found to be most attractive, such as decane, isodecane, isododecane and isohexadecane, and mixtures of various isoparaffins. The preferred solvent is isododecane.

Quenching

After complete polymerization, any unreacted isocyanate group is converted into urethane by adding an alcohol such as ethanol.

Reaction Condition

By balancing the degree of polymerization, the degree of branching, the molecular weight and the percentage of the polyurethane in the solvent solution, the proper viscosity for cosmetic use can be obtained.

The molecular weight can be measured by means of Size Exclusion Chromatography (SEC) or by means of Gel Permeation Chromatography (GPC), a method which uses High Performance Layer Chromatography (HPLC) with an isocratic pump, a refraction index detector and a column thermostat at 25° C. The measurement is performed by dissolving 10-50 mg of the polymer sample in 1 ml THF at room temperature. Subsequently, with a suitable column (e.g. from Polymer Laboratories PL gel) and the calibration curve as function of the expected molecular weight, the chromatographic conditions set up during the calibration phase are maintained (THF mobile phase at room temperature 1 mL/min). Once the test samples are injected, the results are evaluated with suitable GPC software to extrapolate the molecular weight.

The viscosity of a 25-27% by weight solution of the polyurethane of the invention in isododecane can be measured with Brookfield viscosimeter DV-1, with R2 spindle at 20 rpm and at 25° C. For optimum results the polyurethane solution of the invention should have a viscosity of between 1,000 and 10,000, preferably between 1,000 and 6,000, and particularly preferably between 1,300 and 2,500 mPa·s at 25° C. (+/−0.1).

Further Ingredients of the Cosmetic Composition

The composition of the invention may furthermore comprise clays, waxes, solvents, silicones, cosmetic excipients, colourants, preservatives, (co)polymers (other than the polyurethanes of the invention) e.g. polyisoprene, fragrances, flavours, vitamins, antioxidants, vegetable or mineral oils and fats, pearlescent agents, surface-active agents etc. Clays, waxes and solvents are particularly useful further ingredients and are discussed in more detail hereunder.

Clays

The composition of the invention may contain a clay, either unmodified or modified. Typical examples of unmodified clays are smectite clays such as hectorites, montmorillonites and bentonites. Modified clays are clays, which have been made oleophilic by treating them with a cationic compound. Such clays are known in the art. Typical examples are smectite clays such as hectorites, montmorillonites and bentonites, which have been made oleophilic by treating them with an organic cationic compound. Typical examples of the oleophilic modified clays are stearalkonium bentonite, and preferably disteardimonium hectorite. The amount of the clay, when used in the present invention, ranges from 0.05-20% by weight of the composition, and preferably from 0.1-10% by weight.

Solvents

The composition of the invention may contain a solvent It can be any organic solvent, suitable for use in cosmetic products. Typical examples are aliphatic hydrocarbons with from 8 to 24 carbon atoms, such as isoparaffins like isooctane, isononane, isodecane, isododecane, Isopars (RTM) ex Exxon, etc. Isododecane is the preferred solvent.

The solvent can be used in the present invention in an amount of between 1.1-90% by weight of the composition, preferably 10-80% by weight of the composition.

Furthermore, the composition may also preferably contain a silicone, such as (cyclo) polysiloxanes e.g. cyclomethicone and/or dimethicone, in an amount of between 0.5-20% by weight of the composition.

Waxes

The composition of the invention may also comprise a wax, such as candelilla wax, carnauba wax, beeswax, ceresine, microcrystalline wax, paraffin wax, silicone wax, polyethylene wax and the like in an amount of between 0.5-20% by weight of the composition.

Excipients and Colorants

The balance of the composition contains the usual cosmetic excipients, colourants and other additives in an amount of between 1.1%-80%, preferably 5-20% by weight of the composition. Suitable cosmetic excipients are e.g. talc, mica, silicas, kaolin, zinc oxide, calcium carbonate, magnesium carbonate phosphate, starch and its derivatives, nylon, polyethylene, acrylic (co) polymers and so on. Suitable colourants are e.g. iron oxides, chromium oxide and/or hydroxide, blue and pink ultramarine, manganese violet, titanium dioxide, pearlescent pigments based on mica or bismuth oxychloride substrates, carmin lakes and pigments based on organic colorants as listed by the CTFA.

Lipophilic (co)polymers derived from e.g. polyvinylpyrrolidone, from fluor-containing monomers, from acrylic monomers etc, may also be used in the composition of the invention in an amount of between 1-20% by weight of the composition. These lipophilic (co)polymers may even enhance the film-forming action of the polyurethane of the invention.

The composition of the invention may be in liquid, semiliquid, paste-like or cake- or other solid form.

The composition of the invention may be made in any convenient way. A suitable way is first to prepare a dispersion of the polyurethane in the organic solvent, and subsequently adding to the resulting semiliquid mixture the other components of the composition.

The invention will be further illustrated by the following non-limitative examples. The aliquots used in the reactions were at least stoichiometric.

### EXAMPLES

### Example 1

A suitable aliquot of neat siliconic polyol (α,ω-di(2-propoxyethanol)polyd imethylsiloxane (=polydimethylsiloxane, 2-propoxyethanol terminated) was loaded into a stainless steel reactor with stirring. The polyol was diluted with an aliquot of isododecane and the reactor was flushed with nitrogen. The whole reaction was run under nitrogen. A suspension of an aliquot of zinc stearate catalyst in isododecane was added through a dropping funnel. The dropping funnel was rinsed with a suitable aliquot of isododecane which was then added into the reactor. An aliquot of neat IPDI (isophorone diisocyanate) was added into the reactor and the dropping funnel was rinsed with an aliquot of isododecane which was then added into the reactor. The solution was heated at 95° C. under reflux for 15 hours, then cooled at 70° C. and diluted with an aliquot of isododecane. An aliquot of ethyl alcohol was then added and the solution was heated at 80° C. for 3 hours. The absence of free NCO groups was checked by FTIR and the solution was cooled at 50° C. and filtered through a stainless steel sieve. A cloudy solution of the silicone-containing polyurethane in isododecane was obtained. When a neat sample was required, the solvent was removed under reduced pressure and a sticky sample of the crude polyurethane was obtained. The polyurethane had a molecular weight of between 180,000 and 200,000, and a viscosity of between 1,300 and 1,800 mPa·s at a concentration of 25-27% in isododecane and at a temperature of 25° C.

### Example 2

Neat siliconic polyol (same as in example 1) was loaded into a stainless steel reactor under stirring. The polyol was diluted with an aliquot of isododecane and the reactor was flushed under nitrogen. The whole reaction was run under nitrogen. A suspension of an aliquot of zinc stearate catalyst in suitable aliquot of isododecane was added through a dropping funnel. The dropping funnel was rinsed with isododecane which was then added to the reactor. A suitable aliquot of neat IPDI (isophorone diisocyanate) was added to the reactor and the dropping funnel was rinsed with isododecane which was then added to the reactor.

The solution was heated at 95° C. under reflux for 3 hours, and then mixed with a solution of a suitable aliquot of sorbitan monostearate in isododecane which has been previously heated at 95° C. for 30 minutes.

After adding the sorbitan stearate solution, the whole reaction was carried out for 10 hours at 95° C. still under nitrogen reflux.

After 10 hours, a second solution of an aliquot of neat IPDI in isododecane was added to the reactor through the dropping funnel which was then rinsed with isododecane and the reaction was carried out for further 5 hours, still at 95° C. and under nitrogen.

The whole solution was then cooled at 70° C. and suitable aliquot of ethyl alcohol was added to terminate the reaction (to ensure the blocking of isocyanate groups statistically present at one or both ends of the polymer which were then transformed into ethyl urethane) and heated at 80° C. for 3 hours.

The absence of free NCO groups was checked by FTIR and the solution was cooled at 50° C. and filtered through a stainless steel sieve.

A cloudy solution of sugar ester silicone/urethane copolymer in isododecane was obtained, the viscosity of which was in the range of 3,000-4,000 mPa*s at a concentration of 25-27% by weight. The molecular weight was 220,000-260,000. The solvent was removed under reduced pressure and a sticky sample of the crude polyurethane was obtained.

### Example 3

A suitable aliquot of neat siliconic polyol (same as in example 1) was loaded into a stainless steel reactor under stirring. The polyol was diluted with a suitable aliquot of Isododecane and the reactor was flushed under nitrogen. The whole reaction was run under nitrogen. A suspension of a suitable aliquot of zinc stearate catalyst in isododecane was added through a dropping funnel. The dropping funnel was rinsed with an aliquot of isododecane which was then added to the reactor. A suitable aliquot of neat IPDI (isophorone diisocyanate) was added to the reactor and the dropping funnel was rinsed with an aliquot of isododecane which was then added to the reactor.

The solution was heated at 95° C. under reflux for 3 hours, and then mixed with a solution of a suitable aliquot of sorbitan monostearate in isododecane which has been previously heated at 95° C. for 30 minutes.

After adding the sorbitan monostearate solution, the whole reaction Was carried out for 5 hours at 95° C. still under nitrogen reflux.

A second solution of a suitable aliquot of ethyl panthenol in isododecane was added to the reactor through the dropping funnel which was then rinsed with isododecane and the reaction was carried out for further 4 hours, still at 95° C. and under nitrogen.

The whole solution was then cooled at 70° C. and a suitable aliquot of ethyl alcohol was added to terminate the reaction (to ensure the blocking of isocyanate groups statistically present at one or both ends of the polymer which were then transformed into ethyl urethane) and heated at 80° C. for 3 hours.

The absence of free NCO groups was checked by FTIR and the solution was cooled at 50° C. and filtered through a stainless steel sieve.

A cloudy solution of sugar ester panthenol-silicone/urethane copolymer in isododecane was obtained, the viscosity of which was in the range of 4,000-5,000 mPa*s at concentration of about 25-27% by weight. The molecular weight was 280.000 and 320.000. The solvent was removed under reduced pressure and a sticky sample of the crude polyurethane was obtained.

### Example 4

A suitable aliquot of neat siliconic polyol (same as in example 1) was loaded into a stainless steel reactor under stirring. The polyol was diluted with an aliquot of Isododecane and the reactor was flushed under nitrogen. The whole reaction was run under nitrogen. A suspension of a suitable aliquot of zinc stearate catalyst in isododecane was added through a dropping funnel. The dropping-funnel was rinsed with an aliquot of isododecane which was then added to the reactor. A suitable aliquot of neat ethyl lysine diisocyanate was added to the reactor and the dropping funnel was rinsed with isododecane, which was then added to the reactor.

The solution was heated at 95° C. under reflux for 3 hours, and then mixed with a solution of a suitable aliquot of sorbitan monostearate in isododecane, which has been previously heated at 95° C. for 30 minutes.

After adding the sorbitan monostearate solution, the whole reaction was carried out for 10 hours at 95° C. still under nitrogen reflux.

The whole solution was then cooled at 70° C. and a suitable aliquot of ethyl alcohol was added to terminate the reaction (to ensure the blocking of isocyanate groups statistically present at one or both ends of the polymer which were then transformed into ethyl urethane) and heated at 80° C. for 3 hours.

The absence of free NCO groups was checked by FTIR and the solution was cooled at 50° C. and filtered through a stainless steel sieve.

A cloudy solution of sugar ester silicone/urethane copolymer in isododecane was obtained, the viscosity of which was in the range of 3,200-4,200 mPa*s at a concentration of about 25-27% by weight. The molecular weight was 240.000 and 270.000. The solvent was removed under reduced pressure and a sticky sample of the crude polyurethane was obtained.

The following Examples are examples of compositions according to the invention.

### Example 5

### Lip Colouring Fluid

| Ingredient | % weight |
|---|---|
| Isododecane | 61.00 |
| Silicone Polyurethane Ex. 1 | 10.00 |
| Disteardimonium Hectorite | 8.50 |
| Propylene Carbonate | 2.00 |
| Alcohol | 0.80 |
| D&C Red 7 Ca Lake | 0.70 |
| Titanium Dioxide | 1.40 |
| Iron Oxide Yellow | 1.15 |
| Fd&C Blue 1 Al Lake | 0.50 |
| Iron Oxide Red | 0.85 |
| Silica | 0.30 |
| Dimethicone | 10.00 |
| Mica And Titanium Dioxide | 2.60 |
| Flavour | 0.20 |
| TOTAL | 100.00 |

### Example 6

### Lip Colouring Fluid

| Ingredient | % weight |
|---|---|
| Isododecane | 75.45 |
| Silicone Polyurethane Ex. 2 | 8.00 |
| Disteardimonium Hectorite | 6.00 |
| Propylene Carbonate | 1.50 |
| Alcohol | 0.50 |
| D&C Red 7 Ca Lake | 0.70 |
| Titanium Dioxide | 1.40 |
| Iron Oxide Yellow | 1.15 |
| Fd&C Blue 1 Al Lake | 0.40 |
| Iron Oxide Red | 0.85 |
| Silica | 0.30 |
| Mica And Titanium Dioxide | 2.60 |
| Flavour | 0.10 |
| Tocopheryl Linoleate | 0.05 |
| TOTAL | 100.00 |

### Example 7

### Mascara

| Ingredient | % weight |
|---|---|
| Isododecane | 57.0 |
| Cyclomethicone | 10.0 |
| Propylsilsesquioxane | 5.0 |
| Hydrogenated Polyisobutene | 7.0 |
| Silicone Polyurethane Ex. 1 | 3.5 |
| Iron Oxide Black | 7.0 |
| Sucrose Stearate | 1.5 |
| Disteardimonium Hectorite | 0.8 |
| Propylene carbonate | 0.2 |
| TOTAL | 100.0 |

### Example 8

### Lipstick

| Ingredient | % weight |
|---|---|
| Isododecane | 22.95 |
| Polyethylene | 23.00 |
| Cyclopentasiloxane And Polypropylsiloxane | 13.00 |
| Diisostearyl Malate | 3.00 |
| Polymethylsilsesquioxane | 3.50 |
| Silicone Polyurethane Ex. 1 | 6.00 |
| Propylparaben | 0.20 |
| BHT | 0.02 |
| Mica And Titanium Dioxide | 10.20 |
| Iron Oxide Red | 3.40 |
| Iron Oxide Yellow | 0.50 |
| Titanium Dioxide | 1.50 |
| Disteardimonium Hectorite | 0.15 |
| Propylene Carbonate | 0.05 |
| Glycerin | 1.03 |
| Coconut Oil And Tiare' Flower | 0.50 |
| TOTAL | 100.00 |

### Example 9

### Face Fluid Foundation

| Ingredient | % weight |
|---|---|
| Microcrystalline Wax | 1.20 |
| Laureth-9 | 0.55 |
| Polyglyceryl-4 Isostearate | 0.82 |
| Isododecane | 30.43 |
| Silicone Polyurethane Ex. 4 | 5.50 |
| Sodium Chloride | 1.50 |
| Water | 40.00 |
| D&C Red 7 Ca Lake | 0.70 |
| Titanium Dioxide | 8.60 |
| Iron Oxide Yellow | 3.20 |
| Iron Oxide Black | 0.10 |
| Iron Oxide Yellow | 1.40 |
| Iron Oxide Black | 0.30 |
| Iron Oxide Red | 3.50 |
| Glycerine | 2.00 |
| Fragrance | 0.20 |
| TOTAL | 100.00 |

### Example 10

### Powder Eye Shadow

| Ingredient | % weight |
|---|---|
| Isododecane | 2.40 |
| Talc | 59.50 |
| Mica | 4.00 |
| OctylDodecylStearoylStearate | 2.60 |
| Dimethicone | 1.30 |
| Silicone Polyurethane Ex. 1 | 0.80 |
| Iron Oxide Black | 0.10 |
| Iron Oxide Yellow | 8.00 |
| Iron Oxide Red | 3.00 |
| Pearl (Mica/Titanium) | 8.00 |
| Pearl (Mica/TiO2/Iron Oxide) | 10.00 |
| Preservatives | 0.30 |
| TOTAL | 100.0 |

### Example 11

### Compact Powder

| Ingredient | % weight |
|---|---|
| Isododecane | 3.00 |
| Talc | 80.45 |
| Mica | 8.00 |
| OctylDodecylStearoylStearate | 1.70 |
| Dimethicone | 0.80 |
| Silicone Polyurethane Ex. 3 | 1.00 |
| Iron Oxide Black | 0.15 |
| Iron Oxide Yellow | 1.50 |
| Iron Oxide Red | 0.60 |
| Pearl (Mica/Titanium) | 1.00 |
| Nylon 12 | 1.50 |
| Preservatives | 0.30 |
| TOTAL | 100.0 |

1. A cosmetic composition with improved skin substantive, long wear and no-transfer properties comprising:
0.25%-40% by weight of the composition of a particular silicone-containing polyurethane, in which the silicone pre-polymer is an alkoxylated, bis-hydroxyalkyl group terminated polydialkylsiloxane, in which the reactive OH-group is attached to a carbon atom, the silicone-containing polyurethane having a molecular weight of between 5,000 and 800,000.
the balance comprising conventional cosmetic excipients, colourants and additives.

2. A composition according to claim 1, wherein the molecular weight of the silicone-containing polyurethane ranges between 40,000 and 400,000.

3. A composition according to claim 2, wherein the molecular weight of the silicone-containing polyurethane ranges from 100,000 and 280,000.

4. A composition according to claim 1, wherein it comprises from 0.25%-40% of the silicone-containing polyurethane.

5. A composition according to claim 4, wherein it comprises from 0.5%-30% of the silicone-containing polyurethane.

6. A composition according to claim 5, wherein it comprises from 1%-20% of the silicone-containing polyurethane.

7. A composition according to claim 1, wherein the alkoxy group in silicone pre-polymer is a C1-C4 alkoxy group, and the alkoxyl moiety is a C1-C4 alkoxyl moiety.

8. A composition according to claim 7, wherein the alkoxy group is an ethoxy group, and the alkoxyl moiety is a propoxyl moiety.

9. A composition according to claim 1, wherein the silicone-containing polyurethane contains from 1 to 8 alkoxy groups.

10. A composition according to claim 9, wherein the silicone-containing polyurethane contains from 1 to 5 alkoxy groups.

11. A composition according to claim 1, wherein the silicone-containing polyurethane also contains a chain extender.

12. A composition according to claim 11, wherein the chain extender is selected from the group consisting of polyols, C8-C28 fatty acid esters of polyols, hydroxyacids, acylaminoacids, vitamins and vitamin esters.

13. A composition according to claim 12, wherein the chain extender is sorbitan monostearate.

14. A composition according to claim 1, wherein the urethane group is derived from isophorone diisocyanate, lysine ester diisocyanate or arginine ester diisocyanate.

15. A method for preparing a silicone-containing polyurethane, suitable for use in a composition according to claim 1, wherein a silicone pre-polymer according to claim 1 is reacted with a diisocyanate in the presence of a catalyst and a solvent.

16. A method according to claim 15, wherein the catalyst is a zinc salt of a C8-C28 fatty acid, and the solvent a C8-C24 isoparaffin.

17. A method according to claim 15, wherein the reaction product of the silicone pre-polymer and the diisocyanate is subsequently reacted with a chain extender as defined in claim 12 or 13.

18. A method according to claim 17, wherein the chain extender is sorbitan monostearate.

19. Use of a silicone-containing polyurethane as defined in claim 1 as film-forming agent in cosmetic products for the skin, eyes, lips or keratinous materials.
